(19) United States
(12) Patent Application Publication
(10) Pub. No.: US 2017/0266088 A1
(43) Pub. Date: Sep. 21, 2017

(54) SKIN-WHITENING COSMETIC COMPOSITION CONTAINING ROSEMARY-DERIVED VERBENONE AS ACTIVE INGREDIENT

(71) Applicant: Jeju National University Industry-Academic Cooperation Foundation, Jeju-si (KR)

(72) Inventors: Somi KIM, Jeju-si (KR); Deok Hyeon JEON, Jeju-si (KR)

(21) Appl. No.: 15/531,739

(22) PCT Filed: Dec. 1, 2014
(86) PCT No.: PCT/KR2014/011652

§ 371 Date: May 31, 2017

(51) Int. Cl. A61K 8/35; C07C 49/627; A61K 36/53; A61Q 19/02; A61K 31/122

(52) U.S. Cl. CPC A61K 8/35; A61Q 19/02; A61K 31/122; A61K 36/53; C07C 49/627

(57) ABSTRACT

The present invention relates to a skin whitening cosmetic composition comprising verbenone as an active ingredient or a pharmaceutical composition for treating or preventing skin pigmentation disorders.

### TECHNICAL FIELD

The present invention relates to a skin-whitening cosmetic composition containing verbenone as an active ingredient, and more particularly, to a skin-whitening cosmetic composition containing verbenone as an active ingredient or a pharmaceutical composition for treating or preventing skin pigmentation disorders.

### BACKGROUND ART

Skin color is influenced by skin melanin produced from melanocytes, which are pigment cells present in the epidermis, the presence or absence of pigments such as hemoglobin or carotenoids, and the thickness and reflectivity of the skin. In the case of melanin pigment, which is the most important factor for determining skin color, tyrosine, an amino acid normally present in the human body, is converted into 3,4-dihydroxyphenylalanine (DOPA) by tyrosinase, an enzyme present in melanocytes, and the subsequent series of complex oxidation processes ultimately produces melanin, a dark brown polymer. The synthesis of melanin pigment is promoted by factors such as ultraviolet light exposure, melanoma, and hyperpigmentation diseases. In addition, biosynthesis of melanin is regulated by various enzymes including tyrosinase. Among such enzymes, tyrosinase acts on the oxidation of dihydroxyindole after an initial biosynthetic process that converts tyrosine, as a substrate, into L-dopaquinone.

Therefore, studies into finding inhibitors of tyrosinase activity are considered to be important in the development of whitening agents. Currently known tyrosinase inhibitors include hydroquinone, 4-hydroxyanisole, ascorbic acid derivatives, kojic acid, azelaic acid, corticosteroids, retinoids, arbutin, catechin and the like, but use thereof is limited due to safety and economic problems. In recent years, studies on natural products instead of artificial materials, such as raw materials for whitening agent, functional foods and functional cosmetics, using natural plants have been actively conducted in various fields.

Accordingly, a cosmetic composition comprising natural plant extract for alleviating atopic dermatitis is disclosed in Korean Patent No. 10-1236946, and a skin cosmetic composition is disclosed in Korean Patent Application Publication No. 1993-0021190. However, compared to the present invention, a skin whitening cosmetic composition comprising rosemary-derived verbenone as an active ingredient has not been disclosed.

### DISCLOSURE

### Technical Problem

Therefore, the present invention has been made in view of the above problems, and it is an objective of the present invention to provide a skin whitening cosmetic composition comprising verbenone as an active ingredient or a pharmaceutical composition for treating or preventing skin pigmentation disorders. In the present invention, B16F10 melanoma cells, which had been treated with α-melanocyte-stimulating hormone (α-MSH), were treated with rosemary-derived verbenone. As a result, protein expression levels of tyrosinase-related protein-1 (TRP-1), tyrosinase-related protein-2 (TRP-2), tyrosinase, and microphthalmia-associated transcription factor (MITF), which are associated with melanogenesis, were all reduced depending on the concentration of verbenone, and, in addition, melanin content was reduced by verbenone. By confirming these results, the present invention was completed.

### Technical Solution

In accordance with the present invention, the above and other objectives can be accomplished by the provision of a skin whitening cosmetic composition comprising verbenone as an active ingredient.

In accordance with an aspect of the present invention, the above and other objectives can be accomplished by the provision of a pharmaceutical composition for treating or preventing skin pigmentation disorders comprising verbenone as an active ingredient.

### Advantageous Effects

According to the present invention, a skin whitening cosmetic composition comprising rosemary-derived verbenone as an active ingredient or a pharmaceutical composition for treating and improving skin pigmentation disorders can be prepared. In addition, the cosmetic and pharmaceutical compositions of the present invention can effectively inhibit the activity of tyrosinase, an enzyme essential to melanin biosynthesis, and can inhibit melanin biosynthesis in B16F10 melanoma cells. Through these mechanisms, the cosmetic and pharmaceutical compositions have an excellent effect on improving skin whitening and for treating and preventing skin pigmentation disorders.

### DESCRIPTION OF DRAWINGS

FIG. 1 is a graph showing the effects of a rosemary essential oil (a-pinene) and floral waters (1,8-cineole, linalool, camphor, 4-terpineol, and verbenone) on melanin content as active ingredients;

FIG. 2 is a graph showing the inhibitory effect of verbenone on mushroom tyrosinase activity;

FIG. 3 is a graph showing the inhibitory effect of verbenone on tyrosinase activity in B16F10 cells. α-MSH:20 nM α-MSH; Ver:verbenone;

FIG. 4 is a graph showing the effect of verbenone on melanin content in B16F10 cells. α-MSH:20 nM α-MSH; Ver:verbenone;

FIG. 5 is an image showing the colors of B16F10 pellets treated with verbenone. (1) treatment only with 20 nM α-MSH; (2) treatment with 20 nM α-MSH+0.625 mM verbenone; (3) treatment with 20 nM α-MSH+1.25 mM verbenone; (4) treatment with 20 nM α-MSH+2.5 mM verbenone; and

FIG. 6 includes images showing the effect of verbenone on the expression of melanogenesis-related proteins in B16F10 cells. Verbenone was treated at concentrations of 0.625 mM, 1.25 mM, and 2.5 mM respectively; KA, 200 μM kojic acid.

### MODE FOR INVENTION

To achieve the objectives of the present invention, the present invention provides a skin whitening cosmetic composition comprising verbenone as an active ingredient.

In the skin whitening cosmetic composition according to one embodiment of the present invention, the verbenone is preferably derived from rosemary (Rosmarinus officinalis), and more preferably derived from rosemary floral water, without being limited thereto. Floral water is a by-product obtained when extracting essential oils and is also called hydrosol.

In the present invention, “whitening” refers to inhibiting, suppressing or alleviating hyperpigmentation of the skin. Hyperpigmentation of the skin includes freckles, melasma, hyperpigmentation after exposure to ultraviolet light, hyperpigmentation following inflammation, senile lentigines, brown spots or age spots.

In the skin whitening cosmetic composition according to one embodiment of the present invention, skin whitening may be achieved by inhibiting tyrosinase protein expression or melanin production, without being limited thereto.

In the skin whitening cosmetic composition according to one embodiment of the present invention, the content of verbenone may be 0.0001 to 10% by weight based on the total weight of the composition, without being limited thereto.

In the skin whitening cosmetic composition according to one embodiment of the present invention, the cosmetic composition may be variously used to prepare cosmetics and cleansers having a skin whitening effect and, in particular, may have a formulation selected from the group consisting of a skin ointment, a cream, an emollient, a nutrition toner, a face pack, an essence, a hair tonic, a shampoo, a hair rinse, a hair conditioner, a hair treatment, a gel, a skin lotion, a skin softener, a skin toner, an astringent, a lotion, a milk lotion, a moisture lotion, a nutrition lotion, a massage cream, a nutrition cream, a moisture cream, a hand cream, a foundation, a nutrition essence, a sunscreen, a soap, a cleansing foam, a cleansing lotion, a cleansing cream, a body lotion and a body cleanser, without being limited thereto.

When the formulation of the present invention is a paste, cream or gel, animal fibers, plant fibers, wax, paraffin, starches, tragacanth, cellulose derivatives, polyethylene glycol, silicone, bentonite, silica, talc or zinc oxides may be used as a carrier component.

When the formulation of the present invention is a powder or spray, lactose, talc, silica, aluminum hydroxide, calcium silicate or polyamide powders may be used as a carrier component, and, in particular, in the case of a spray, the formulation may additionally include propellants such as chlorofluorohydrocarbons, propane/butane or dimethyl ether.

When the formulation of the present invention is a solution or emulsion, solvents, solvating agents or emulsifying agents may be used as a carrier component. For example, water, ethanol, isopropanol, ethyl carbonate, ethyl acetate, benzyl alcohol, benzyl benzoate, propylene glycol, 1,3-butyl glycol oil, glycerol aliphatic esters, polyethylene glycol or fatty acid sorbitan esters may be used.

When the formulation of the present invention is a suspension, liquid diluents such as water, ethanol or propylene glycol, suspensions such as ethoxylated isostearyl alcohol, polyoxyethylene sorbitol esters and polyoxyethylene sorbitan esters, microcrystalline cellulose, aluminum metahydroxide, bentonite, agar or tragacanth may be used as a carrier component.

When the formulation of the present invention is a surfactant-containing cleanser, aliphatic alcohol sulfates, aliphatic alcohol ether sulfates, sulfosuccinic acid monoesters, isethionates, imidazolinium derivatives, methyl taurates, sarcosinates, fatty acid amide ether sulfates, alkylamidobetaines, aliphatic alcohols, fatty acid glycerides, fatty acid diethanolamides, vegetable oils, lanolin derivatives or ethoxylated glycerol fatty acid esters may be used as a carrier component.

The formulation of the present invention may additionally contain excipients including fluorescent substances, fungicides, hydrotropic substances, moisturizers, fragrances, fragrance carriers, proteins, solubilizing agents, sugar derivatives, sunscreens, vitamins, plant extracts and the like.

When a skin whitening cosmetic composition is formulated into a pharmaceutical product or a cosmetic as described above, the composition may contain a known excipient that acts as a carrier for an active ingredient and is applicable to the skin. When the skin whitening cosmetic composition is formulated into a pharmaceutical product, the contents described in “Remington's Pharmaceutical Science, Mack Publishing Company, Easton Pa.” may be referenced. When the skin whitening cosmetic composition is formulated into a cosmetic, the contents described in “International Cosmetic Ingredient Dictionary, 6th ed., The Cosmetic, Toiletry and Fragrance Association, Inc., Washington, 1995” may be referenced.

In addition, the present invention provides a pharmaceutical composition for treating or preventing skin pigmentation disorders comprising verbenone as an active ingredient.

In the pharmaceutical composition for treating or preventing skin pigmentation disorders according to one embodiment of the present invention, the verbenone is preferably derived from rosemary (Rosmarinus officinalis), and is more preferably derived from rosemary floral water, without being limited thereto.

In the pharmaceutical composition for treating or preventing skin pigmentation disorders according to one embodiment of the present invention, the skin pigmentation disorders may locally occur on skin due to increased synthesis of melanin pigment, and may be one or more disorders selected from melasma, freckles, lentigines, nevi, drug-induced pigmentation, pigmentation following inflammation, and hyperpigmentation caused by dermatitis, without being limited thereto.

In the pharmaceutical composition for treating or preventing skin pigmentation disorders according to one embodiment of the present invention, treatment or prevention of the skin pigmentation disorders may be achieved by inhibiting tyrosinase protein expression or melanin production, without being limited thereto.

In the pharmaceutical composition for treating or preventing skin pigmentation disorders according to one embodiment of the present invention, the content of verbenone may be 0.0001 to 10% by weight based on the total weight of the composition, without being limited thereto.

In the pharmaceutical composition for treating or preventing skin pigmentation disorders according to one embodiment of the present invention, the pharmaceutical composition may be formulated into cream, gel, patch, spray, ointment, plaster, lotion, liniment, paste or cataplasma, without being limited thereto.

In addition, the present invention provides a food composition or beverage composition for skin whitening comprising verbenone as an active ingredient.

When verbenone of the present invention is used as a food additive, verbenone may be added alone or in combination with other foods or food ingredients, and may be suitably used according to conventional methods. The amount of the active ingredient to be mixed may be suitably determined depending on the intended use (prevention, health or therapeutic treatment). In general, in the manufacture of food or beverages, 15 parts by weight or less, preferably 10 parts by weight or less, of verbenone of the present invention may be added per 100 parts by weight of raw material. However, in the case of long-term ingestion intended for health and hygiene purposes or for health control purposes, the amount may be less than the above range. Since the active ingredient has no issues in terms of safety, the active ingredient may be used in the above-mentioned range or more.

There is no particular limitation on the type of the food. For example, by using verbenone, it is possible to produce processed food having excellent storage properties while utilizing the characteristics of agricultural products, livestock products or aquatic products. Such processed food includes, for example, confectioneries, drinks, alcoholic beverages, fermented food, canned food, processed milk products, processed meat products, and noodles. The confectioneries include biscuits, pies, breads, candies, jellies, gums, and cereals (including meal substitution foods such as cereal flakes). The drinks include carbonated beverages, functional ionic beverages, juices (such as apple, pear, grape, aloe, citrus fruit, peach, carrot, tomato juices, etc.), sikhye, and the like. The alcoholic beverages include rice wine, whiskey, soju, beer, liquor, fruit wine, and the like. The fermented foods include soy sauce, soybean paste, red pepper paste, and the like. The canned foods include canned marine products (e.g., tuna, mackerel, saury pike, canned conch, etc.), canned livestock products (canned products such as beef, pork, chicken, and turkey) and canned agricultural products (canned products such as corn, peaches and pineapples). The processed milk products include cheese, butter, yogurt, and the like. The processed meat products include pork cutlets, beef cutlets, chicken cutlets, sausage, sweet and sour pork, nuggets, marinated grilled beef slices, and the like. The noodles include sealed and packaged noodles and the like. In addition, the composition may be used in retort food, soup and the like.

In addition, verbenone may be used to produce functional foods, health foods or health supplements. The functional foods, health foods or health supplements refer to foods that provide biological control functions by including bioactive ingredients, in addition to nutritional functions. Since verbenone of the present invention has an effect of improving skin whitening, verbenone may be used for the production of functional foods, health foods or health supplements.

In addition, the present invention provides a method of skin whitening, the method comprising applying verbenone and a pharmaceutically or cosmetically acceptable carrier on a subject with skin pigmentation disorders in need of skin whitening.

In the method according to one embodiment of the present invention, the verbenone is preferably derived from rosemary (Rosmarinus officinalis), and more preferably derived from rosemary floral water, without being limited thereto.

In the method according to one embodiment of the present invention, the skin pigmentation disorders may locally occur on skin due to increased synthesis of melanin pigment, and may be one or more disorders selected from melasma, freckles, lentigines, nevi, drug-induced pigmentation, pigmentation following inflammation, and hyperpigmentation caused by dermatitis, without being limited thereto.

In the method according to one embodiment of the present invention, the method of skin whitening may be achieved by inhibiting tyrosinase protein expression or melanin production, without being limited thereto.

In a method according to one embodiment of the present invention, the content of verbenone may be 0.0001 to 10% by weight based on the total weight of the composition, without being limited thereto.

In the method according to one embodiment of the present invention, the pharmaceutical composition for treating or preventing skin pigmentation disorders may be formulated into cream, gel, patch, spray, ointment, plaster, lotion, liniment, paste or cataplasma, without being limited thereto.

In addition, the present invention provides a method of treating or preventing skin pigmentation disorders, the method comprising applying verbenone and a pharmaceutically or cosmetically acceptable carrier on a subject in need of treatment or prevention of skin pigmentation disorders.

In the method according to one embodiment of the present invention, the verbenone is preferably derived from rosemary (Rosmarinus officinalis), and more preferably derived from rosemary floral water, without being limited thereto.

In the method according to one embodiment of the present invention, the skin pigmentation disorders may locally occur on skin due to increased synthesis of melanin pigment, and may be one or more disorders selected from melasma, freckles, lentigines, nevi, drug-induced pigmentation, pigmentation following inflammation, and hyperpigmentation caused by dermatitis, without being limited thereto.

In the method according to one embodiment of the present invention, treatment or prevention of the skin pigmentation disorders may be achieved by inhibiting tyrosinase protein expression or melanin production, without being limited thereto.

In a method according to one embodiment of the present invention, the content of verbenone may be 0.0001 to 10% by weight based on the total weight of the composition, without being limited thereto.

In the method according to one embodiment of the present invention, the pharmaceutical composition for treating or preventing skin pigmentation disorders may be formulated into cream, gel, patch, spray, ointment, plaster, lotion, liniment, paste or cataplasma, without being limited thereto.

When the formulation of the present invention is a paste, cream or gel, animal fibers, plant fibers, wax, paraffin, starches, tragacanth, cellulose derivatives, polyethylene glycol, silicone, bentonite, silica, talc or zinc oxides may be used as a carrier component.

When the formulation of the present invention is a powder or spray, lactose, talc, silica, aluminum hydroxide, calcium silicate or polyamide powders may be used as a carrier component, and, in particular, in the case of a spray, the formulation may additionally include propellants such as chlorofluorohydrocarbons, propane/butane or dimethyl ether.

When the formulation of the present invention is a solution or emulsion, solvents, solvating agents or emulsifying agents may be used as a carrier component. For example, water, ethanol, isopropanol, ethyl carbonate, ethyl acetate, benzyl alcohol, benzyl benzoate, propylene glycol, 1,3-butyl glycol oil, glycerol aliphatic esters, polyethylene glycol or fatty acid sorbitan esters may be used.

When the formulation of the present invention is a suspension, liquid diluents such as water, ethanol or propylene glycol, suspensions such as ethoxylated isostearyl alcohol, polyoxyethylene sorbitol esters and polyoxyethylene sorbitan esters, microcrystalline cellulose, aluminum metahydroxide, bentonite, agar or tragacanth may be used as a carrier component.

When the formulation of the present invention is a surfactant-containing cleanser, aliphatic alcohol sulfates, aliphatic alcohol ether sulfates, sulfosuccinic acid monoesters, isethionates, imidazolinium derivatives, methyl taurates, sarcosinates, fatty acid amide ether sulfates, alkylamidobetaines, aliphatic alcohols, fatty acid glycerides, fatty acid diethanolamides, vegetable oils, lanolin derivatives or ethoxylated glycerol fatty acid esters may be used as a carrier component.

The formulation of the present invention may additionally contain excipients including fluorescent substances, fungicides, hydrotropic substances, moisturizers, fragrances, fragrance carriers, proteins, solubilizing agents, sugar derivatives, sunscreens, vitamins, plant extracts and the like.

When a skin whitening cosmetic composition is formulated into a pharmaceutical product or a cosmetic as described above, the composition may contain a known excipient that acts as a carrier for an active ingredient and is applicable to the skin. When the skin whitening cosmetic composition is formulated into a pharmaceutical product, the contents described in “Remington's Pharmaceutical Science, Mack Publishing Company, Easton Pa.” may be referenced. When the skin whitening cosmetic composition is formulated into a cosmetic, the contents described in “International Cosmetic Ingredient Dictionary, 6th ed., The Cosmetic, Toiletry and Fragrance Association, Inc., Washington, 1995” may be referenced.

Hereinafter, the present invention is described in detail with reference to examples. However, the following examples are provided for illustrative purposes only and should not be construed as limiting the scope and spirit of the present invention.

### Materials and Experimental Methods

### 1. Materials

The stems and leaves of rosemary (Rosmarinus officinalis) grown at the Urban Farmers Farm in Pyoseonmyeon, Seogwipo, Jeju Island were collected in summer 2012 and used after drying. In addition, all reagents were purchased from Sigma Chemical Co. (St. Louis, Mo., USA) and Invitrogen/Gibco Co. (Grand Island, N.Y., USA).

### 2. Measurement of melanin content

B16F10 melanoma cells, obtained from mice, were used as melanin-producing cells. The cultured B16F10 cells were treated with 20 M α-melanocyte-stimulating hormone (α-MSH) (a control group). After 1 day, 2.5, 1.25, 0.625 mM verbenone and 2 mM arbutin (a positive control group) were added to the cells, the cells were cultured for 1 day and 2 days, respectively (experimental groups). The cultured cells were washed twice with PBS and harvested by centrifugation at 1500 rpm for 5 minutes. 300 μl of 1 N NaOH containing 10% DMSO was added to the cell pellet and then incubated at 80° C. for 1 hour to dissolve melanin. Melanin content was calculated by the following equation.

Melanin content (%)=A/B×100

A: OD490 value of treated group

B: OD490 value of non-treated group

### 3. Measurement of mushroom tyrosinase inhibition activity

60 μl of a 1 M phosphate buffer (pH 6.8), 10 μl of a sample solution, and 20 μl of 500 U/ml mushroom tyrosinase were sequentially added, and then incubated at room temperature for 5 minutes. After incubation, 60 μl of an aqueous solution containing 1 mM L-tyrosine was added to initiate a reaction. The reaction was performed at 37° C. for 30 minutes, and absorbance was measured at 490 nm. Mushroom tyrosinase inhibition activity was calculated by the following equation.

Mushroom tyrosinase inhibition activity (%)=(B−A)/A×100

A: OD490 value of treated group

B: OD490 value of blank control

### 4. Measurement of Tyrosinase Inhibition Activity in B16F10 Cells

Cultured cells were harvested by adding tyrosine-EDTA. After harvesting, 500 μl of a cell lysis buffer (10 mM sodium phosphate buffer containing 1% Triton X-100 and 0.1 mM PMSF) was added to the harvested cells. To lyse the cells, sonication was performed on the cells and subsequently the cells were incubated on ice for 30 minutes. The lysed cells were centrifuged at 13000 rpm for 30 minutes, and the obtained cell lysate was used to measure tyrosinase activity.

### 5. Western Blot Analysis

B16F10 cells (5×10³ cells/μl) were treated with 20 nM α-MSH. After 24 hours, the cells were treated with verbenone at concentrations of 0.625, 1.25, and 2.5 mM, respectively, followed by culturing for 72 hours. The cultured cells were harvested and washed three times with PBS. Then, to lyse the cells, 500 μl of a lysis buffer was added to the cells and the cells were sonicated, followed by incubation for 30 minutes. After cell lysis, centrifugation was performed at 12,000 rpm and 4° C. for 30 minutes to obtain a supernatant. Protein concentration was quantified using bovine serum albumin (BSA) as a standard. Equivalent amounts of the denatured lysates were loaded onto a 12% sodium dodecyl sulfate-polyacrylamide gel, and electrophoresis was performed at 45 mA to separate proteins. After sodium dodecyl sulfate-polyacrylamide gel electrophoresis (SDS-PAGE), the separated proteins present in the gel were transferred onto a membrane at 150 V for 80 minutes. Then, blocking of the membrane was performed for overnight using TTBS (TBS+0.1% Tween 20) solution containing 5% skim milk. Primary antibodies against melanogenesis-related proteins and actin were prepared in a ratio of 1:1,000, and a primary antibody reaction was performed at room temperature for 3 hours. Anti-rabbit IgG and anti-mouse IgG were used as secondary antibodies and diluted 1:5,000. A secondary antibody reaction was performed at room temperature for 1 hour. After the antibody reaction, the membrane was washed three times with a TTBS solution, and then the membrane was exposed to an X-ray film after a reaction with an ECL substrate for 1 to 3 minutes.

### Example 1. Selection of Whitening Active Ingredient of Rosemary Produced in Jeju

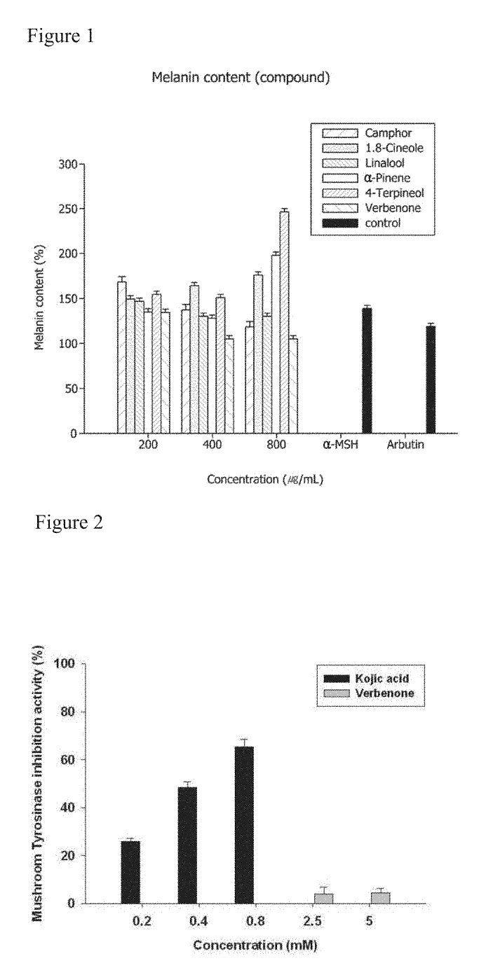

To identify a whitening active material from essential oil and floral waters of rosemary produced in Jeju, B16F10 mouse melanoma cells were treated with six major components contained in essential oil (α-pinene) and floral waters (1,8-cineole, linalool, camphor, 4-terpineol, and verbenone) of rosemary produced in Jeju with various concentrations. After 72 hours, the change in melanin content in B 16F10 cells was measured at a wavelength of 490 nm. As a result, melanin content was most significantly decreased in the verbenone-treated cells compared to the control group, arbutin-treated cells. Therefore, verbenone, which had not yet been reported, was focused upon and subsequent experiments were conducted (FIG. 1).

### Example 2. Whitening Efficacy of Verbenone

### \textcircled{1} Evaluation of Whitening Efficacy by Measuring Mushroom Tyrosinase Inhibition Activity

Using verbenone selected by screening the ingredients of rosemary produced in Jeju, a mushroom tyrosinase inhibition assay was performed. When kojic acid used as a control was treated at a concentration of 200, 400 or 800 μM, the effect of kojic acid on inhibiting tyrosinase activity increased by treatment of 20 nM α-MSH was observed in a concentration-dependent manner. On the other hand, no inhibitory effect was observed in the groups treated with 2.5 and 5 mM verbenone (FIG. 2). This data indicates that verbenone does not act as an enzyme inhibitor against tyrosinase.

### \textcircled{2} Evaluation of Whitening Efficacy by Tyrosinase Inhibition Assay

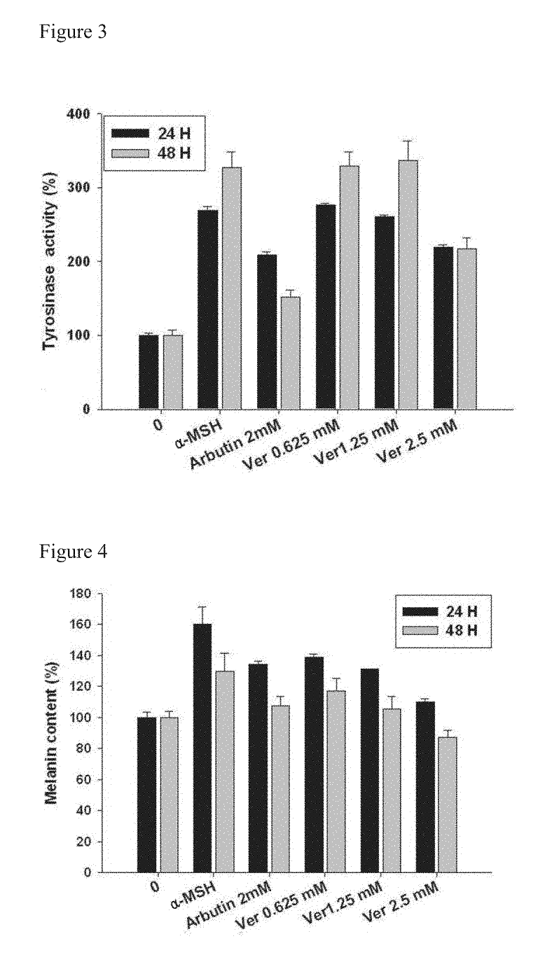

To evaluate the inhibitory efficacy of verbenone against tyrosinase in B16F10 mouse melanoma cells, B16F10 cells were treated with α-MSH for one day to increase tyrosinase. Then, the cells were treated with verbenone at concentrations of 0.625, 1.25 and 2.5 mM, respectively, and tyrosinase activity was measured after 24 and 48 hours, respectively. As a result, it was confirmed that verbenone inhibited tyrosinase in B16F10 cells (FIG. 3).

These results and the results of in vitro mushroom tyrosinase inhibition assay suggest that verbenone exhibits a whitening effect by inhibiting the expression of tyrosinase in cells rather than acting as an enzyme inhibitor of tyrosinase.

### \textcircled{3} Evaluation of Whitening Efficacy by Melanin Content Analysis

To assess the ability of verbenone to reduce melanin content in B16F10 cells, B 16F10 cells were treated with α-MSH and incubated for 24 hours to increase melanin content. After incubation, the cells were treated with verbenone at concentrations of 0.625, 1.25 and 2.5 mM, respectively, and melanin content was measured after 24 and 48 hours, respectively. As a result, it was confirmed that melanin content was reduced by verbenone in B16F10 cells in a concentration-dependent manner (FIG. 4). In addition, when the color of the cell pellet was examined, the color of the pellet was also weakened by verbenone in a concentration-dependent manner (FIG. 5).

### \textcircled{4} Measurement of Level of Melanogenesis-Related Proteins Using Western Blotting

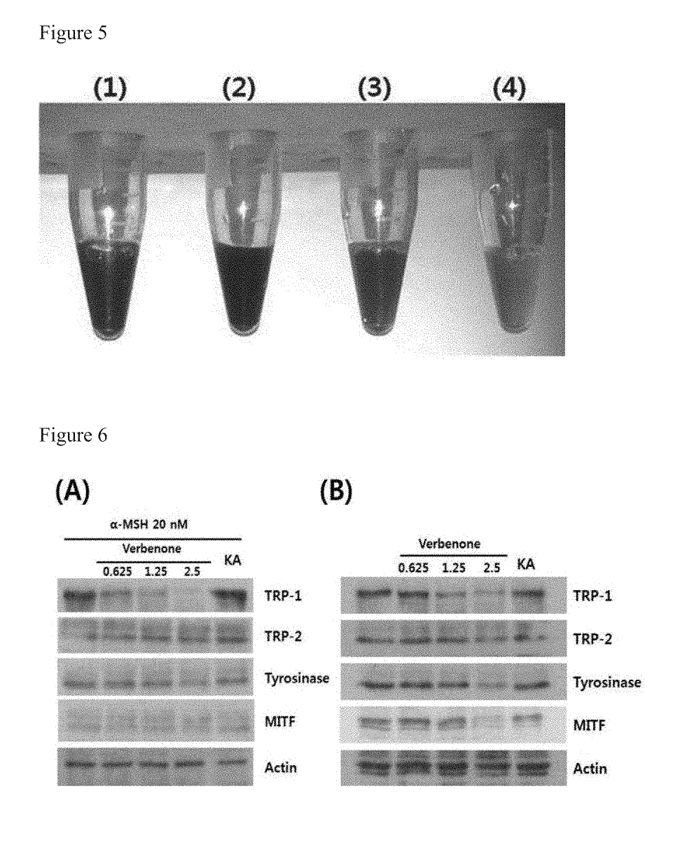

To confirm the mechanism of the whitening effect of verbenone, expression levels of melanogenesis-related proteins, such as tyrosinase-related protein 1 (TRP-1), tyrosinase-related protein 2 (TRP-2), tyrosinase and microphthalmia-associated transcription factor (MITF), were measured using western blotting. B16F10 cells were treated with verbenone and kojic acid as a control, respectively. After 72 hours, when the expression levels of melanogenesis-related proteins TRP-1, TRP-2, tyrosinase and MITF were measured, the expression levels of all of the above proteins were decreased by verbenone in a concentration-dependent manner (FIG. 6B). In addition, B16F10 cells were treated with 20 nM α-MSH for 24 hours, and then the cells were treated with verbenone and kojic acid, respectively. Thereafter, when the expression levels of the melanogenesis-related proteins were examined, the expression levels of the proteins were reduced in the verbenone-treated group in a concentration-dependent manner (FIG. 6A).

1. A skin whitening cosmetic composition comprising verbenone as an active ingredient.

2. The composition according to claim 1, wherein the verbenone is derived from rosemary.

3. The composition according to claim 1, wherein skin whitening is achieved by inhibiting tyrosinase protein expression or melanin production.

4. The composition according to claim 1, wherein the content of the verbenone is 0.0001 to 10% by weight based on the total weight of the composition.

5. The composition according to claim 1, wherein the composition is a formulation selected from the group consisting of a skin ointment, a cream, an emollient, a nutrition toner, a face pack, an essence, a hair tonic, a shampoo, a hair rinse, a hair conditioner, a hair treatment, a gel, a skin lotion, a skin softener, a skin toner, an astringent, a lotion, a milk lotion, a moisture lotion, a nutrition lotion, a massage cream, a nutrition cream, a moisture cream, a hand cream, a foundation, a nutrition essence, a sunscreen, a soap, a cleansing foam, a cleansing lotion, a cleansing cream, a body lotion and a body cleanser.

6. A pharmaceutical composition for treating or preventing skin pigmentation disorders comprising verbenone as an active ingredient.

7. The composition according to claim 6, wherein the verbenone is derived from rosemary.

8. The composition according to claim 6, wherein the skin pigmentation disorders locally occur on skin due to increased synthesis of melanin pigment, and are one or more disorders selected from melasma, freckles, lentigines, nevi, drug-induced pigmentation, pigmentation following inflammation, and hyperpigmentation caused by dermatitis.

9. The composition according to claim 6, wherein treatment or prevention of the skin pigmentation disorders are achieved by inhibiting tyrosinase protein expression or melanin production.

10. The composition according to claim 6, wherein the content of the verbenone is 0.0001 to 10% by weight based on the total weight of the composition.

11. The composition according to claim 6, wherein the composition is formulated into cream, gel, patch, spray, ointment, plaster, lotion, liniment, paste or cataplasma.

12. A food composition for skin whitening comprising verbenone as an active ingredient.

13. (canceled)

14. A method of skin whitening, the method comprising applying verbenone and a pharmaceutically or cosmetically acceptable carrier on a subject with skin pigmentation disorders in need of skin whitening.

15. The method according to claim 14, wherein the verbenone is derived from rosemary.

16. The method according to claim 14, wherein the skin pigmentation disorders locally occur on skin due to increased synthesis of melanin pigment, and are one or more disorders selected from melasma, freckles, lentigines, nevi, drug-induced pigmentation, pigmentation following inflammation, and hyperpigmentation caused by dermatitis.

17. A method of treating or preventing skin pigmentation disorders, the method comprising applying verbenone and a pharmaceutically or cosmetically acceptable carrier on a subject in need of treatment or prevention of skin pigmentation disorders.

18. The method according to claim 17, wherein the verbenone is derived from rosemary.

19. The method according to claim 17, wherein the skin pigmentation disorders locally occur on skin due to increased synthesis of melanin pigment, and are one or more disorders selected from melasma, freckles, lentigines, nevi, drug-induced pigmentation, pigmentation following inflammation, and hyperpigmentation caused by dermatitis.
